(19) United States
(12) Patent Application Publication
(10) Pub. No.: US 2008/0171779 A1
(43) Pub. Date: Jul. 17, 2008

(54) USE OF 5-HT6 ANTAGONISTS TO PREVENT RELAPSE INTO ADDICTION

(75) Inventors: Natasja M.W.J. De Bruin, Weesp (NL); Arnold Van Loevezijn, Weesp (NL); Johan Wijnen, Weesp (NL); Arnoldus H.J. Herremans, Weesp (NL); Cornelis G. Kruse, Weesp (NL)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Solvay Pharmaceuticals B.V.

(21) Appl. No.: 12/013,898

(22) Filed: Jan. 14, 2008

Related U.S. Application Data
(60) Provisional application No. 60/880,421, filed on Jan. 16, 2007.

(51) Int. Cl. A61K 31/415; A61P 25/30

(52) U.S. Cl. 514/403

(57) ABSTRACT

A novel use of compounds and pharmaceutically acceptable salts thereof, which are 5-HT₆ antagonists, are disclosed. In one embodiment, the invention relates to the use of these compounds or pharmaceutical compositions comprising these compounds for preventing relapse into addiction, for example, relapse into addiction to substances of abuse, including opiates, hallucinogens, inhalants, phencyclidine, amphetamines, cocaine, cannabis, nicotine, and alcohol, relapse into addiction to certain medicines, including sedatives, hypnotics and anxiolytics, and relapse into certain addictive behaviors, including gambling.

This application claims the benefit of priority of U.S. Provisional Application No. 60/880,421, filed on Jan. 16, 2007, the disclosure of which is incorporated herein by reference.

Embodiments of the invention relate to a novel use of 5-HT₆ antagonists and pharmaceutically acceptable salts thereof such as the use of these compounds or pharmaceutical compositions comprising these compounds for preventing relapse into addiction or addictive behaviors.

Addiction is caused by a combination of genetic, drug-induced, and environmental factors, and is often considered a chronic relapsing disease. Many times it is. But, fortunately, not always. “Addiction”, physical and/or psychical dependence, can be the result of the use of a variety of substances of abuse, such as opiates, hallucinogens, cocaine, cannabis, nicotine and alcohol, often termed “drugs”. This sometimes causes confusion with “real” drugs, also known as medicines. It should be realized however, that the distinction between “real” drugs and other “drugs” is vague. When used in appropriate concentrations opiates, cocaine and cannabis certainly have therapeutic benefits. Mutatis mutandis: when used improperly, medicines, in particular certain sedatives, hypnotics and anxiolytics, certainly can become addictive. For many people, running, shopping, gambling, smoking, and drinking, are enjoyable pastimes. Yet, these pastimes, in particular the latter three, often result in addiction.

There is no established therapy that can prevent addiction except for discipline. Psychotherapy, as well as a number of different medicines, to a different extent can be successful to overcome an addiction. When a former addict is no longer compulsively seeking the drug, does not crave it anymore, or experience its withdrawal symptoms, the addiction is over, and that person can no longer be considered to be a patient. This happens, but unfortunately not always. Once an addict is ‘clean’ the story ends, at least as far as pharmacological intervention is concerned. There are no drugs clinically proven to prevent relapse.

It is generally known that even after prolonged periods of drug abstinence, vulnerability to relapse for prior addicts is high. Relapse can be triggered by re-exposure to the drug (De Wit, 1996), by re-exposure to drug-associated stimuli (Childress, 1992) or by exposure to stressors (Sinha, 1999). Alcohol is one of the most widely and frequently abused addictive drugs in the world. In laboratory animals it is possible to study relapse to alcohol seeking in a so-called “reinstatement model”. In this model, by using similar conditions that trigger relapse in the human situation, it is possible to reinstate alcohol seeking in animals after prolonged periods of withdrawal (Liu, 2002; Lê, 1998). In the reinstatement model, De Vries et al. observed that the CB₁-receptor antagonist SR141716A (rimonabant) is able to selectively reduce relapse behavior induced by conditioned drug cues previously associated with cocaine, heroin, nicotine and alcohol delivery (De Vries, 2001, 2003, 2005). This indicates a possible therapeutic role for CB₁-antagonists in preventing relapse into addiction to substances of abuse.

5-HT₆ antagonists have been suggested to be of value in treating drug and alcohol addiction (alcoholism), and in treating withdrawal from drug abuse, specifically: alcohol, cocaine, nicotine, and benzodiazepines (GB 2,341,549, US 2003/0220325, US 2005/020575, US 2005/020596, U.S. Pat. No. 6,187,805, WO 01/017963, WO 02/041889, WO 02/051832, WO 02/051837, WO 02/060903, WO 2002/089811, WO 2002/098857, WO 2003/013510, WO 2003/053433, WO 2003/066632, WO 2003/072548, WO 2003/072558, WO 2003/089438, WO 2003/099797, WO 2004/009600, WO 2005/066157 and WO 2006/081332). However, there are no indications that 5-HT₆ antagonists are potential therapeutics for preventing relapse into addiction.

A goal of the present invention is to develop drugs for preventing relapse into addiction, drugs that have a mechanism of action different from antagonism of cannabinoid-CB₁ receptors.

### DESCRIPTION OF EMBODIMENTS OF THE INVENTION

Surprisingly, 5-HT₆ antagonists were found to be active in an animal model predictive of relapse behavior in humans: stimulus induced reinstatement of extinguished self-administration behavior in rats (Shaham, 2003). After learning to consume alcohol 12% (v/v) in a two-bottle paradigm, rats were trained to self-administer alcohol in the operant cage. When response-rates were stable, animals were withdrawn from alcohol for three weeks. Subsequently, the effect of 5-HT₆ antagonists on relapse was tested by re-exposing the animals to alcohol-related stimuli. It was found that these compounds were able to suppress cue-induced alcohol-seeking behavior.

Using a very similar test protocol, 5-HT₆ antagonists were also found to suppress cue-induced nicotine-seeking behavior. Thus 5-HT₆ antagonists are believed useful for the preparation of medicaments for preventing relapse into addiction, in particular relapse into addiction to substances of abuse, including opiates, hallucinogens, inhalants, phencyclidine, amphetamines, cocaine, cannabis, nicotine, and alcohol, medicaments for preventing relapse to addiction to certain medicines, including sedatives, hypnotics and anxiolytics, and medicaments for preventing relapse to certain addictive behaviors, including gambling.

In one embodiment, the invention embraces the use of 5-HT₆ antagonists for preventing relapse to addiction, the 5-HT₆ antagonists being chosen from the following: BGC-20-761, BVT-74316, CNS-10000, CNS-11010, CNS-25100, diF-BAMPI, E-6837, FMPD, GSK-215083, JCF-177, KR-055014, KR-055015, LY-483518(=SGS-518), MS-245, PHA-565272A, PRX-07034, Ro 04-6790, Ro-4368554, Ro 63-0563, Ro 65-7199, Ro 65-7674, Ro 66-0074, SAM-315, SB-214111, SB-258510, SB-258585, SB-271046, SB-331711, SB-357134, SB-399885, SB-699929, SB-737050A, SB-742457(=GSK-742457), ST-1938, and WAY-181187(=SAX-187).

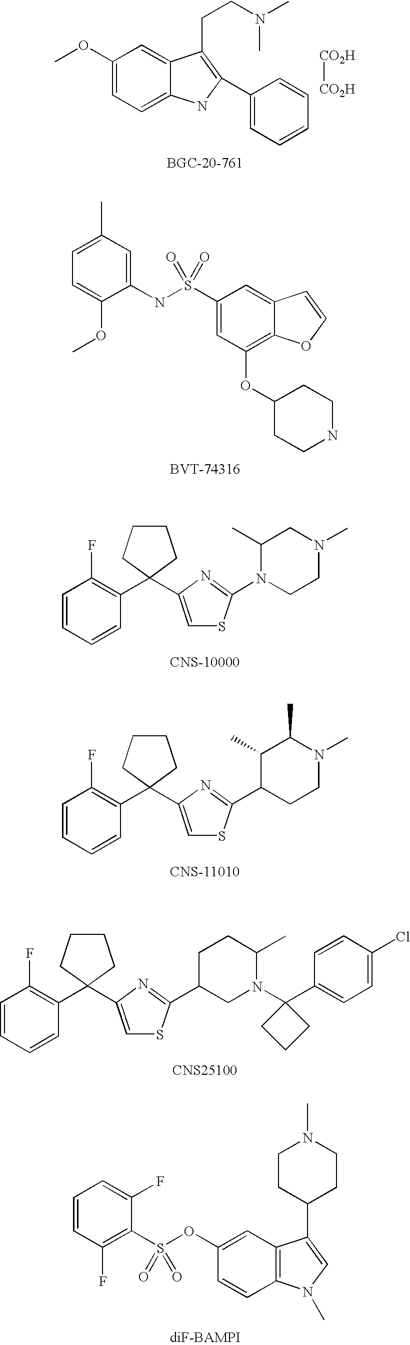

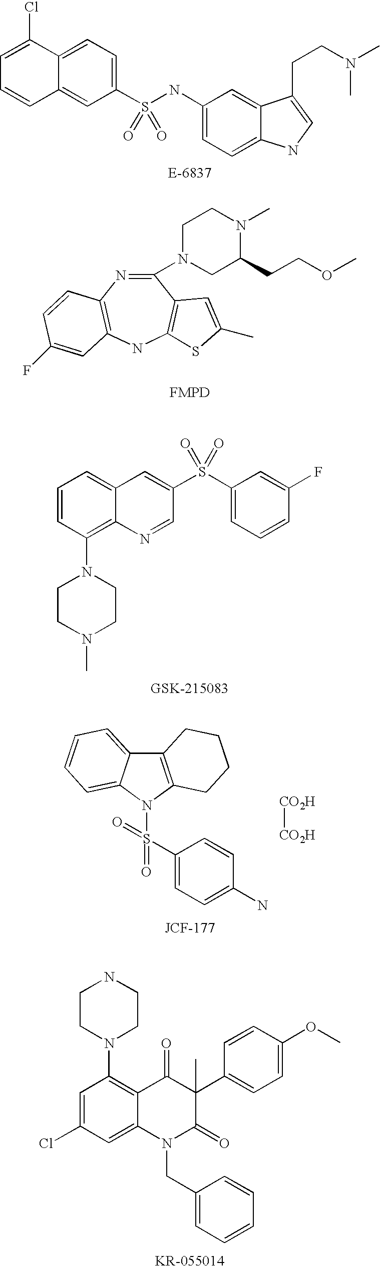

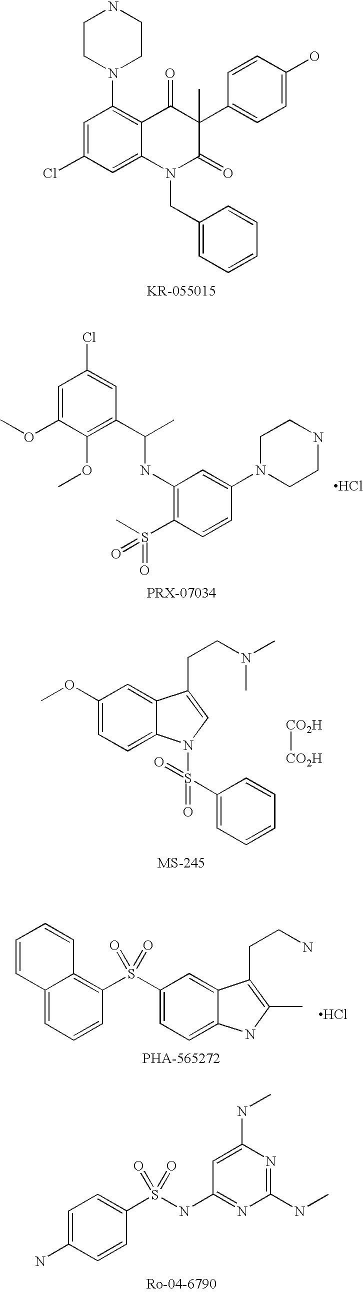

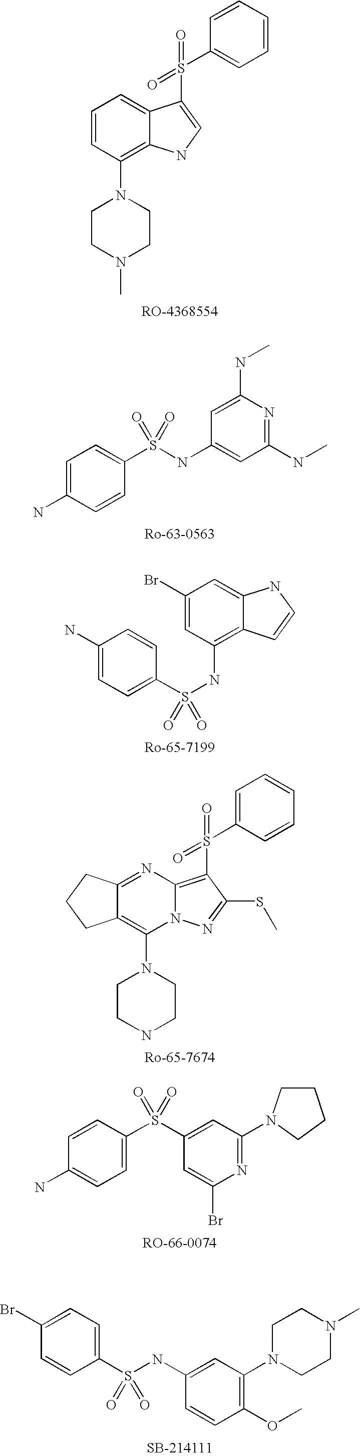

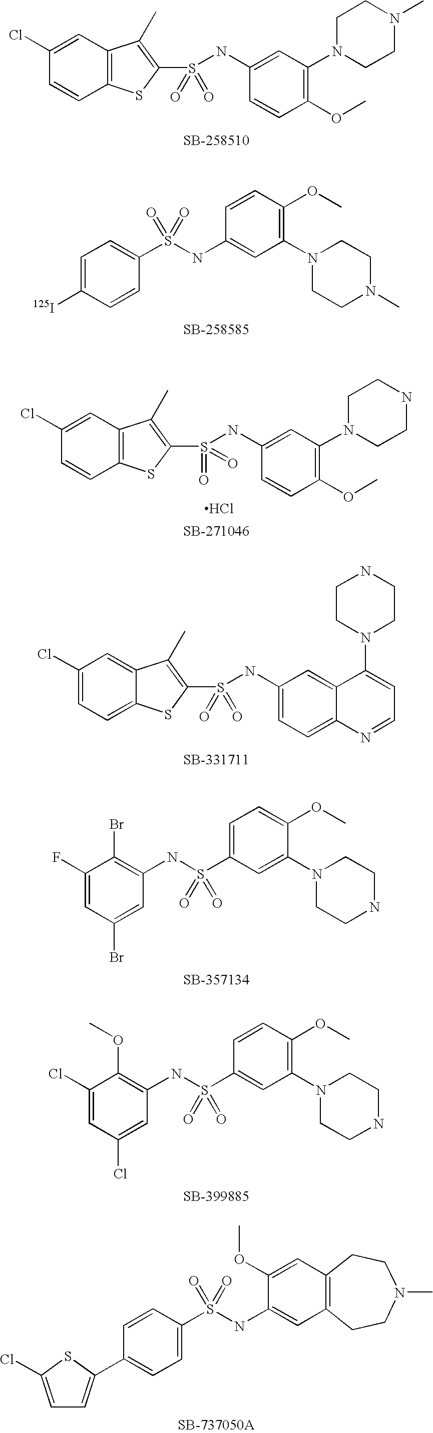

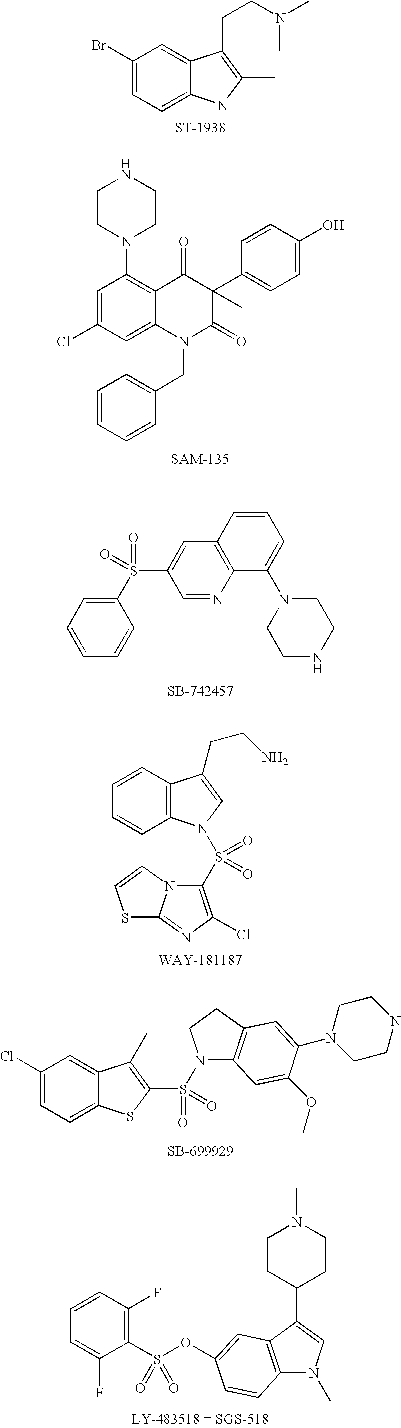

In other embodiments, the invention relates to the use of 5-HT₆ antagonists for preventing relapse into addiction wherein the 5-HT₆ antagonists are described in the following patents and patent applications: EP 0 815 861, DE 10053794, DE 10053795, DE 10053796, DE 10053799, DE 10053813, GB 2,341,549, US 2003/220325, US 2003/229069, US 2004/019064, US 2005/124613, US 2005/020575, US 2005/020596, US 2006/069094, US 2006/084676, U.S. Pat. No. 5,990,105, U.S. Pat. No. 6,133,287, U.S. Pat. No. 6,187,805, U.S. Pat. No. 6,191,141, U.S. Pat. No. 6,194,410, U.S. Pat. No. 6,943,169, WO 1998/027058, WO 1998/027081, WO 1999/002502, WO 1999/037623, WO 1999/042465, WO 1999/047516, WO 1999/065906, WO 2000/012073, WO 2000/012623, WO 2000/034242, WO 2000/037452, WO 2000/063203, WO 2001/012629, WO 2001/017963, WO 2001/032646, WO 2001/032660, WO 2001/038316, WO 2002/008178, WO 2002/008179, WO 2002/018358, WO 2002/032863, WO 2002/036115, WO 2002/041889, WO 2002/042293, WO 2001/017963, WO 2002/041889, WO 2002/051832, WO 2002/051837, WO 2002/060871, WO 2002/060903, WO 2002/078693, WO 2002/089811, WO 2002/092585, WO 2002/098857, WO 2002/100822, WO 2002/100842, WO 2003/013510, WO 2003/014097, WO 2003/035061, WO 2003/039547, WO 2003/042175, WO 2003/053433, WO 2003/066056, WO 2003/066632, WO 2003/068220, WO 2003/068732, WO 2003/068740, WO 2003/068752, WO 2003/072198, WO 2003/072548, WO 2003/072558, WO 2003/080580, WO 2003/082877, WO 2003/089438, WO 2003/099792, WO 2003/099797, WO 2003/101962, WO 2003/101990, WO 2003/104193, WO 2004/000828, WO 2004/009548, WO 2004/009600, WO 2004/014895, WO 2004/026830, WO 2004/031181, WO 2004/035047, WO 2004/041781, WO 2004/041792, WO 2004/048328, WO 2004/050085, WO 2004/078176, WO 2004/080969, WO 2004/098588, WO 2005/014578, WO 2005/016891, WO 2005/021530, WO 2005/025576, WO 2005/026177, WO 2005/037834, WO 2005/051397, WO 2005/051398, WO 2005/051399, WO 2005/058858, WO 2005/066157, WO 2005/067933, WO 2005/095346, WO 2005/105776, WO 2005/113539, WO 2006/037481, WO 2006/037482, WO 2006/038006, WO 2006/061126, WO 2006/062481, WO 2006/066745, WO 2006/066746, WO 2006/066748, WO 2006/066756, WO 2006/066790, WO 2006/081332, WO 2006/091703, WO 2006/114402 and WO 2006/134150.

In yet other embodiments, the invention relates to the use of one or more compounds of formula (1) for preventing relapse into addiction, wherein formula (1) is:

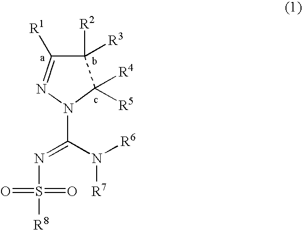

or a tautomer, stereoisomer, N-oxide, or isotopically-labelled analogue of the foregoing, or a pharmacologically acceptable salt, hydrate or solvate of any of the foregoing, wherein:
R₁ is chosen from a hydrogen atom, an unsubstituted alkyl(C₁₋₄) group, and an alkyl(C₁₋₄) group substituted with one or more halogen atoms;
R₂ and R₃ are independently chosen from a hydrogen atom, an unsubstituted alkyl(C₁₋₄) group, and an alkyl(C₁₋₄) group substituted with one or more halogen atoms, or,
R₁ and R₂, together with the carbon atoms marked ‘a’ and ‘b,’ form a C₅₋₈-cycloalkyl ring, or
R₂ and R₃, together with the carbon atom marked ‘b,’ form a C₃₋₈-cycloalkyl ring;
the dotted line between the carbon atoms marked ‘b’ and ‘c’ represents either a single or a double bond;
R₄ and R₅ are independently chosen from a hydrogen atom, an unsubstituted alkyl(C₁₋₄) group, and an alkyl(C₁₋₄) group substituted with one or more halogen atoms, or, R₃ and R₄, together with the carbon atoms marked ‘b’ and ‘c,’ form a C₃₋₈-cycloalkyl ring, or
R₄ and R₅, together with the carbon atom marked ‘c,’ form a C₃₋₈-cycloalkyl ring;
R₆ and R₇ are independently chosen from a hydrogen atom, an alkyl(C₁₋₄) group, an alkyl(C₁₋₄) group substituted with one or more halogen atoms, a (C₁₋₃)alkoxy group, a dialkyl(C₁₋₃)amino-alkyl(C₁₋₃) group, an optionally substituted aryl group, an optionally substituted C₅₋₈-cyclo-alkyl group, and an optionally substituted C₅₋₈-heterocycloalkyl group, or
R₆ and R₇, together with the nitrogen atom to which they are attached, form an optionally substituted C₅₋₈-heterocycloalkyl group; and
R₈ is chosen from an optionally substituted aryl group, and a —CR₉═CR₁₀-aryl group,
wherein R₉ and R₁₀ are independently chosen from a hydrogen atom, an alkyl-(C₁₋₃) group, and a —C≡C-aryl group.

In one embodiment, the invention relates to the use of racemates, mixtures of diastereomers as well as individual stereoisomers of the compounds having formula (1). In another embodiment, the invention relates to the use of the E isomer, Z isomer and E/Z mixtures of compounds having formula (1).

Compounds of formula (1), their synthesis, physicochemical properties, affinity for 5-HT₆ receptors (pKᵢ-values), and functional activity as antagonists (pA₂ values) on 5-HT₆ receptors, were disclosed in PCT/EP2007/059944, filed on Sep. 20, 2007, the disclosure of which is incorporated by reference herein.

Further embodiments of the invention relate to the use of one or more compounds of formula (1) or a tautomer, stereoisomer, N-oxide, or isotopically-labelled analogue thereof, or a pharmacologically acceptable salt, hydrate or solvate of any of the foregoing, for preventing relapse into addiction, wherein in formula (1):
R₁ is a hydrogen atom, or R₁ and R₂, together with the carbon atoms marked ‘a’ and ‘b,’ form a cyclohexyl ring;
R₂ and R₃ are independently chosen from a hydrogen atom, and an alkyl(C₁₋₃) group, or R₂ and R₃, together with the carbon atom marked ‘b,’ form a cyclopentyl or cyclohexyl ring;
the dotted line between the carbon atoms marked ‘b’ and ‘c,’ represents either a single or a double bond;
R₄ and R₅ are independently chosen from a hydrogen atom and an alkyl(C₁₋₃) group, or
R₃ and R₄, together with the carbon atoms marked ‘b’ and ‘c’ form a C₃₋₈-cycloalkyl ring;
R₆ and R₇ are independently chosen from a hydrogen atom, an alkyl(C₁₋₃) group, an alkyl(C₁₋₄) group substituted with one or more halogen atoms, a methoxy group, a cyclohexyl group, a benzyl group, and a 4-piperidinyl group; and
R₈ is chosen from an optionally substituted aryl group, and an —CR₉═CR₁₀-aryl group, wherein R₉ and R₁₀ are independently chosen from a hydrogen atom, an alkyl(C₁₋₃) group, and a —C≡C-aryl group.

In another embodiment the invention relates to the use of compounds of formula (1) or a tautomer, stereoisomer, N-oxide, or isotopically-labelled analogue thereof, or a pharmacologically acceptable salt, hydrate or solvate of any of the foregoing, for preventing relapse into addiction, wherein in formula (1), each of R₁, R₄, R₅ and R₆ is a hydrogen atom, R₂ and R₃ are independently an alkyl(C₁₋₃) group, or R₂ and R₃, together with the carbon atom marked ‘b,’ form a cyclopentyl, or cyclohexyl ring, the dotted line between the carbon atoms marked ‘b’ and ‘c’ represents a single bond, R₇ is an alkyl(C₁₋₃) group, and R₈ is a mono- or bicyclic aryl group, substituted with one or more halogen atoms.

In one embodiment, the invention relates to the use of the compound of formula (1) for preventing relapse into addicition, wherein the compound of formula (1) is ‘compound 33’ disclosed in PCT/EP2007/059944, having the following structure:

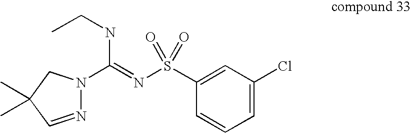

Other embodiments of the invention include, but are not limited to:

methods for preventing relapse into addiction to substances of abuse, to medicines, or to addictive behaviors;

methods for preventing relapse into addiction to substances of abuse, chosen from opiates, hallucinogens, inhalants, phencyclidine, amphetamines, cocaine, cannabis, nicotine, and alcohol;

methods for preventing relapse into addiction to alcohol;

methods for preventing relapse into addiction to nicotine;

methods for preventing relapse into addiction to cannabis;

methods for preventing relapse into addiction to opiates;

methods for preventing relapse into addiction to cocaine;

methods for preventing relapse into addiction to medicines chosen from sedatives, hypnotics and anxiolytics; and

methods for preventing relapse into addiction to gambling.

### DEFINITIONS

Within the context of this description, the term “5-HT₆ receptor antagonist” refers to a compound capable of displaying this activity—measured by unambiguous and well accepted pharmacological assays, including those described in PCT/EP2007/059944—without displaying substantial cross-reactivity towards another receptor. In one embodiment, a compound of the present invention is at least 10 times more potent as a 5-HT₆ receptor antagonist than as an agonist or antagonist on any other receptor. In other embodiments, a compound of the present invention has a 100-fold selectivity, or, for example, a selectivity of a factor of 1,000 or higher.

General terms used in the description of compounds herein disclosed bear their usual meanings. The term alkyl as used herein denotes a univalent saturated, branched or straight, hydrocarbon chain. Unless otherwise stated, such chains can contain from 1 to 18 carbon atoms. Representative of such alkyl groups are methyl, ethyl, propyl, isopropyl, butyl, isobutyl, sec-butyl, tert-butyl, pentyl, isopentyl, neopentyl, tert-pentyl, hexyl, isohexyl, heptyl, octyl, nonyl, decyl, undecyl, dodecyl, tridecyl, tetradecyl, pentadecyl, hexadecyl, heptadecyl, octadecyl, and the like. When qualified “lower”, the alkyl group will contain from 1 to 6 carbon atoms. The same carbon content applies to the parent term “alkane”, and to derivative terms such as “alkoxy”. The carbon content of various hydrocarbon containing moieties is indicated by a prefix designating the minimum and maximum number of carbon atoms in the moiety, i.e., the prefix Cₓ-C_{y} defines the number of carbon atoms present from the integer “x” to the integer “y” inclusive. “Alkyl(C₁₋₃)” for example, means methyl, ethyl, n-propyl or isopropyl, and “alkyl(C₁₋₄)” means methyl, ethyl, n-propyl, isopropyl, n-butyl, 2-butyl, isobutyl or 2-methyl-n-propyl. The term “alkenyl” denotes straight or branched hydrocarbon radicals having one or more carbon-carbon double bonds, such as vinyl, allyl, butenyl, etc., and for example represents (C₂₋₄)alkenyl. In “alkynyl” groups, the straight or branched hydrocarbon radicals have one or more carbon-carbon triple bonds, such as ethynyl, propargyl, 1-butynyl, 2-butynyl, etc., and for example represent (C₂₋₄)alkynyl. Unless otherwise stated, “alkenyl” and “alkynyl” chains can contain from 1 to 18 carbon atoms.

The term “acyl” means alkyl(C₁₋₃) carbonyl, arylcarbonyl or aryl-alkyl(C₁₋₃)carbonyl. “Aryl” embraces monocyclic or fused bicyclic aromatic or hetero-aromatic groups, including but not limited to furyl, thienyl, pyrrolyl, oxazolyl, thiazolyl, imidazolyl, imidazo[2,1-b][1,3]thiazolyl, pyrazolyl, isoxazolyl, isothiazolyl, pyridyl, pyridazinyl, pyrimidinyl, pyrazinyl, 1,3,5-triazinyl, phenyl, indazolyl, indolyl, indolizinyl, isoindolyl, benzo[b]furanyl, 1,2,3,4-tetrahydro-naphtyl, 1,2,3,4-tetrahydroisoquinolinyl, indanyl, indenyl, benzo[b]thienyl, 2,3-dihydro-1,4-benzodioxin-5-yl, benzimidazolyl, benzothiazolyl, benzo[1,2,5]thia-diazolyl, purinyl, quinolinyl, isoquinolinyl, phtalazinyl, quinazolinyl, quinoxalinyl, 1,8-naphthyridinyl, naphthyl, pteridinyl or azulenyl. “Halo” or “Halogen” means chloro, fluoro, bromo or iodo. “Hetero” as in ‘heteroalkyl, heteroaromatic’ etc. means containing one or more N, O or S atoms. “Heteroalkyl” includes alkyl groups with heteroatoms in any position, thus including N-bound O-bound or S-bound alkyl groups.

The term “substituted” means that the specified group or moiety bears one or more substituents. Where any group may carry multiple substituents, and a variety of possible substituents is provided, the substituents are independently selected, and need not be the same. The term “unsubstituted” means that the specified group bears no substituents. With reference to substituents, the term “independently” means that when more than one of such substituents are possible, they may be the same or different from each other.

“Optionally substituted” means that a group may or may not be further substituted by one or more groups selected from C₁₋₈ alkyl, C₁₋₈ alkenyl, C₁₋₈ alkynyl, aryl, fluoro, chloro, bromo, hydroxyl, C₁₋₈ alkyloxy, C₁₋₈ alkenyloxy, aryloxy, acyloxy, amino, C₁₋₈ alkylamino, dialkyl(C₁₋₈)-amino, arylamino, thio, C₁₋₈alkylthio, arylthio, cyano, oxo, nitro, acyl, amido, C₁₋₈ alkylamido, dialkyl(C₁₋₈)amido, and carboxyl, or two optional substituents may together with the carbon atoms to which they are attached form a 5- or 6-membered aromatic or non-aromatic ring containing 0, 1 or 2 heteroatoms selected from nitrogen, oxygen or sulphur. Optional substituents may themselves bear additional optional substituents. These optional substituents may include C₁₋₃ alkyl, such as for example methyl, ethyl, and trifluoromethyl, fluoro, chloro, bromo, hydroxyl, C₁₋₃ alkyloxy such as for example methoxy, ethoxy and trifluoromethoxy, and amino.

“C₃₋₈-cycloalkyl” means cyclopropyl, cyclobutyl, cyclopentyl, cyclohexyl, cyclopheptyl or cyclooctyl. “C₅₋₈ heterocycloalkyl” refers to heteroatom containing rings including but not limited to piperidinyl, morpholinyl, azepanyl, pyrrolidinyl, thiomorpholinyl, piperazinyl, tetrahydrofuryl, tetrahydropyranyl;

The terms “oxy”, “thio” and “carbo” as used herein as part of another group respectively refer to an oxygen atom, a sulphur atom and a carbonyl (C═O) group, serving as linker between two groups, such as for instance hydroxyl, oxyalkyl, thioalkyl, carboxyalkyl, etc. The term “amino” as used herein alone, or as part of another group, refers to a nitrogen atom that may be either terminal, or a linker between two other groups, wherein the group may be a primary, secondary or tertiary (two hydrogen atoms bonded to the nitrogen atom, one hydrogen atom bonded to the nitrogen atom and no hydrogen atoms bonded to the nitrogen atom, respectively) amine. The terms “sulfinyl” and “sulfonyl” as used herein as part of another group respectively refer to an —SO— or an —SO₂— group.

To provide a more concise description, the terms “compound” or “compounds” include tautomers, stereoisomers, N-oxides, and isotopically-labelled analogues, as well as pharmacologically acceptable salts, hydrates and solvates of any of the foregoing, when not explicitly mentioned.

N-oxides of the compounds mentioned above belong to the invention. Tertiary amines may or may not give rise to N-oxide metabolites. The extent to what N-oxidation takes place varies from trace amounts to a near quantitative conversion. N-oxides may be more active than their corresponding tertiary amines, or less active. While N-oxides can easily be reduced to their corresponding tertiary amines by chemical means, in the human body this happens to varying degrees. Some N-oxides undergo nearly quantitative reductive conversion to the corresponding tertiary amines, in other cases conversion is a mere trace reaction, or even completely absent (Bickel, 1969).

Any compound metabolized in vivo to provide the bioactive agent (i.e., the compound of formula (1)) is a prodrug within the scope and spirit of the application. Prodrugs are therapeutic agents, inactive per se but transformed into one or more active metabolites. Thus, in the methods of treatment of the present invention, the terms “administering” or “use in the treatment of” shall encompass treating the various disorders described with the compound specifically disclosed, or with a compound that not specifically disclosed, but that converts to the specified compound in vivo after administration to the patient. Prodrugs are bioreversible derivatives of drug molecules used to overcome some barriers to the utility of the parent drug molecule. These barriers include, but are not limited to, solubility, permeability, stability, presystemic metabolism and targeting limitations (Bundgaard, 1985; King, 1994; Stella, 2004; Ettmayer, 2004; Järvinen, 2005). Prodrugs, i.e., compounds that when administered to humans or mammals by any known route, are metabolized to compounds having formula (1), are within the scope of the invention. In particular, this relates to compounds with primary or secondary amino or hydroxy groups. Such compounds can be reacted with organic acids to yield compounds having formula (1) wherein an additional group is present that is easily removed after administration, for instance, but not limited to amidine, enamine, a Mannich base, a hydroxyl-methylene derivative, an O-(acyloxymethylene carbamate) derivative, carbamate, ester, amide or enaminone.

“Solvates” are generally a crystal form that contains either stoichiometric or non-stoichiometric amounts of a solvent. Often, during the process of crystallization some compounds have a tendency to trap a fixed molar ratio of solvent molecules in the crystalline solid state, thus forming a solvate. When the solvate is water, “hydrates” may be formed. The compound of formula (1) and pharmaceutically acceptable salts thereof may exist in the form of a hydrate or a solvate, and such a hydrate and solvate are also encompassed in the present invention. Examples thereof include ¼ hydrate, dihydrochloride dihydrate, and the like. “Amorphous” forms are noncrystalline materials with no long range order, and generally do not give a distinctive powder X-ray diffraction pattern. Crystal forms in general have been described by Byrn (1995) and Martin (1995).

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

The terms “selective” and “selectivity” refer to compounds that display reactivity towards a particular receptor (e.g., a 5-HT₆ receptor) without displaying substantial cross-reactivity towards another receptor. Thus, for example, selective compounds of the present invention may display reactivity towards 5-HT₆ receptors without displaying substantial cross-reactivity towards other 5-HT receptors. In one embodiment, a compound of the present invention has at least about 10-fold selectivity to the 5-HT₆ receptor, at least about 50-fold selectivity to the 5-HT₆ receptor, at least about 100-fold selectivity to the 5-HT₆ receptor, at least about 250-fold selectivity to the 5-HT₆ receptor, or at least about 500-fold selectivity to the desired target.

Throughout the description and the claims of this specification the word “comprise” and variations of the word, such as “comprising” and “comprises” is not intended to exclude other additives, components, integers or steps.

While it may be possible for the compounds of formula (1) to be administered as the raw chemical, these compounds are typically administered as a “pharmaceutical composition”. According to a further aspect, the present invention provides a pharmaceutical composition comprising at least one compound of formula (1), at least one pharmaceutically acceptable salt or solvate thereof, or a mixture of any of the foregoing, together with one or more pharmaceutically acceptable carriers thereof, and optionally one or more other therapeutic ingredients. The carrier(s) must be “acceptable” in the sense of being compatible with the other ingredients of the formulation and not deleterious to the recipient thereof. The term “composition” as used herein encompasses a product comprising specified ingredients in predetermined amounts or proportions, as well as any product that results, directly or indirectly, from combining specified ingredients in specified amounts. In relation to pharmaceutical compositions, this term encompasses a product comprising one or more active ingredients, and an optional carrier comprising inert ingredients, as well as any product that results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. The pharmaceutical composition includes enough of the active object compound to produce the desired effect upon the progress or condition of diseases. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

Dose, the affinity of the compounds of the invention for 5-HT₆ receptors, was determined as described below. From the binding affinity measured for a given compound of formula (1), one can estimate a theoretical lowest effective dose. At a concentration of the compound equal to twice the measured Kᵢ-value, nearly 100% of the 5-HT₆ receptors likely will be occupied by the compound. Converting that concentration to mg of compound per kg of patient yields a theoretical lowest effective dose, assuming ideal bioavailability. Pharmacokinetic, pharmaco-dynamic, and other considerations may alter the dose actually administered to a higher or lower value. The typical daily dose of the active ingredients varies within a wide range and will depend on various factors such as the relevant indication, the route of administration, the age, weight and sex of the patient, and may be determined by a physician. In general, total daily dose administration to a patient in single or individual doses, may be in amounts, for example, from 0.001 to 10 mg/kg body weight daily, and more usually from 0.01 to 1,000 mg per day, of total active ingredients. Such dosages will be administered to a patient in need of treatment from one to three times each day, or as often as needed for efficacy, and for periods of at least two months, more typically for at least six months, or chronically.

The term “therapeutically effective amount” as used herein refers to an amount of a therapeutic agent to treat a condition treatable by administrating a composition of the invention. That amount is the amount sufficient to exhibit a detectable therapeutic or ameliorative response in a tissue system, animal or human. The effect may include, for example, treating the conditions listed herein. The precise effective amount for a subject will depend upon the subject's size and health, the nature and extent of the condition being treated, recommendations of the treating physician (researcher, veterinarian, medical doctor or other clinician), and the therapeutics, or combination of therapeutics, selected for administration. Thus, it is not useful to specify an exact effective amount in advance. The term “pharmaceutically acceptable salt” refers to those salts that are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well-known in the art. They can be prepared in situ when finally isolating and purifying the compounds of the invention, or separately by reacting them with pharmaceutically acceptable non-toxic bases or acids, including inorganic or organic bases and inorganic or organic acids (Berge, 1977). The “free base” form may be regenerated by contacting the salt with a base or acid, and isolating the parent compound in the conventional matter. The parent form of the compound differs from the various salt forms in certain physical properties, such as solubility in polar solvents, but otherwise the salts are equivalent to the parent form of the compound for the purposes of the present invention. “Complex” refers to a complex of the compound of the invention, e.g., formula (1), complexed with a metal ion, where at least one metal atom is chelated or sequestered. Complexes are prepared by methods well known in the art (Dwyer, 1964).

The term “treatment” as used herein refers to any treatment of a mammalian, for example human condition or disease, and includes: (1) inhibiting the disease or condition, i.e., arresting its development, (2) relieving the disease or condition, i.e., causing the condition to regress, or (3) stopping the symptoms of the disease. The term “inhibit” includes its generally accepted meaning which includes prohibiting, preventing, restraining, alleviating, ameliorating, and slowing, stopping or reversing progression, severity, or a resultant symptom. As such, the present method includes both medical therapeutic and/or prophylactic administration, as appropriate. As used herein, the term “medical therapy” is intended to include prophylactic, diagnostic and therapeutic regimens carried out in vivo or ex vivo on humans or other mammals. “Mammals” include animals of economic importance such as bovine, ovine, and porcine animals, especially those that produce meat, as well as domestic animals, sports animals, zoo animals, and humans. The term “subject” as used herein, refers to an animal, such as a mammal, for example, a human, who has been the object of treatment, observation or experiment.

### EXAMPLES

### Stimulus Induced Reinstatement of Extinguished Self-Administration of Alcohol

Animals: 64 male Wistar rats, weighing 280-300 g at arrival (Harlan, the Netherlands) were used for testing compound 33. The animals were housed individually in a temperature—(T=22±2° C.) and humidity (rel %=60±5%) controlled room on a reversed 12 h light-dark cycle (lights off at 7:00 AM) with water available ad libitum. The animals were on a diet of 18 gr chow per animal per day. The experiment started after one week of acclimatization and lasted 12-14 weeks.

Drug Self-administration: All training and testing was conducted in 16 operant chambers (30.5×24.1×29.2 cm) surrounded by sound attenuating ventilated cubicles (Med Associates, Georgia, Vt.). The chambers were fitted with a grid floor, a red dim house light and opposite the house light an intelligence panel. In the intelligence panel there were two nose-poke holes (Ø2.5 cm) located 5.5 cm above the grid floor and 16 cm separated from each other. A red dim stimulus light was located 12 cm above both nose-poke holes. Between the two nose-poke holes, a receptacle was located, in which the alcohol was delivered. High above the receptacle was a sonalert tone module. One nose-poke hole served as active- and the other as inactive hole. Pokes made in the active hole resulted in the delivery of an alcohol drop (0.19 ml; 12%) in the receptacle. The alcohol drop was delivered by an infusion pump (PHM-100, MedAssociates, Georgia, Vt.) in 4.2 seconds During alcohol delivery, the active nose-poke hole was illuminated and a tone (±65 dB) was presented by the sonalert tone module for 2 seconds. The receptacle was illuminated for 5 seconds (discrete cues). The stimulus light switched off for 15 seconds (discriminative cue). In this 15 second time-out period, nose-poking was without consequences. During all sessions, responses in the inactive hole were monitored, but without consequences.

Acquisition & withdrawal: The animals were trained on a Fixed Ratio 1 schedule (FR1-schedule) for 10 daily sessions (every active response resulted in a reward; sessions of 60 min). Subsequently, animals were trained via an FR2-schedule up to an FR4-schedule (every 2^{nd} and 4^{th} active response respectively resulted in a reward; daily sessions of 60 min). Animals were selected for further testing if they consumed alcohol doses (in the FR4-schedule) above the literature described pharmacological effective dose of 0.3 g/kg/hr (Linseman, 1987). Subsequently, when response behavior on the FR4-schedule was stable, there was a 3 week period of withdrawal in the homecage.

Test for reinstatement: After the withdrawal period, each animal was tested twice for cue induced reinstatement with a week of further extinction training and wash-out in between. Animals were randomly distributed over the different experimental groups and never received the same treatment twice. On test days, the animals were placed in the operant cage for three or four hours (test day 1: 4 hr; test day 2: 3 hr) in absence of alcohol and alcohol related stimuli (discrete and discriminative cues) to extinguish response behavior. One hour before the end of this extinction session, the animal was removed from the operant cage and treated with a 5-HT₆ antagonist. After drug administration the animals was placed back into the operant chamber, where the session continued. When the last hour had passed, the cue-induced relapse test session started (duration: 45 min). During this test, the experimental conditions were similar to the conditions during training on an FR4-schedule, with the exceptions that 1) at the beginning of the session, a 12% alcohol drop of 0.20 ml was available (delivered in the receptacle by the experimenter at the begin of the test session) and 2) after every 4^{th} response, only cues were presented (no alcohol was delivered). Tests were operated and raw data was saved by the MED-PC IV program (Tatham, 1989).

Drugs: Alcohol 12% (v/v) was made from alcohol 96% and diluted with tap water. Compound 33 was suspended in 5% Tween 80, 0.25% methylcellulose, 5% PEG 400 and H₂O HPLC (65% of end volume). Ethanol was added and evaporated under N₂-gas. The pH was adjusted to 5.8. In the range: 0, 3,10, and 30 mg/kg, compound 33 was administered intraperitoneally, 15 minutes before testing. The injection volume was 2 ml/kg.

Statistical analysis: For analysis of the cue-induced reinstatement data, the total number of nose-pokes (responses) in the active and inactive hole were subjected to a one-way ANOVA, followed by a Fisher's LSD Multiple-Comparison Test, with the different doses of 5-HT₆ antagonist as between factor. Because each animal was tested twice for cue-induced reinstatement and cue-reactivity, the results were usually lower during the second test; test day was used as a covariate.

### Stimulus Induced Reinstatement of Extinguished Self-Administration of Nicotine

Animals: 32 male Wistar rats, weighing 280-300 g at arrival (Harlan, the Netherlands) were used for testing compound 33. The animals were housed individually in a temperature—(T=22±2° C.) and humidity (rel %=60±5%) controlled room on a reversed 12 h light-dark cycle (lights off at 7:00 AM) with water and food available ad libitum. The experiment started after one week of acclimatization and lasted 12-14 weeks.

Surgery: after a week of acclimatization, intravenous silicon catheters are implanted in the right jugular vein under isoflurane anesthesia as described previously in De Vries, 1999.

Drug Self-administration: All training and testing was conducted in 16 operant chambers (30.5×24.1×29.2 cm) surrounded by sound attenuating ventilated cubicles (Med Associates, Georgia, Vt.). The chambers were fitted with a grid floor, a red dim house light and opposite the house light an intelligence panel. In the intelligence panel there were two nose-poke holes (Ø2.5 cm) located 5.5 cm above the grid floor and 16 cm separated from each other. A red dim stimulus light was located 12 cm above both nose-poke holes. A tone module was located at the top of the center of the intelligence panel. During self-administration, animals were connected to an infusion pump (PHM-100, Med Associates, Georgia, V.T.) by a swivel to receive intravenous infusions of nicotine (40 μg/kg/inf). One nose poke hole served as active- and the other as inactive hole. Pokes made in the active hole resulted in the delivery of a nicotine infusion for 2 seconds. During this infusion, the active nose poke hole was illuminated (yellow light) and an audio stimulus was presented (tone of 2900 Hz and 65 dB for 2 seconds) (discrete cues). From the moment of nicotine delivery, the stimulus light switched off for 15 seconds (discriminative cue). In this time-out period, nose poking was without consequences. During all sessions, responses in the inactive hole were monitored, but without consequences.

Acquisition, withdrawal and reinstatement: Behavioral training started 5-7 days after surgery. The animals were trained on a Fixed Ratio 1 schedule (FR1-schedule) for 10 daily sessions (every active nose poke resulted in a nicotine infusion and associated cues; daily sessions of 60 min). Subsequently, animals were trained on an FR2-schedule for three days and then moved up to an FR3-schedule (respectively, every 2nd and 3^{rd} active nose poke resulted in a reward and associated cues; daily sessions of 60 min). When response behavior was stable, animals were selected for further testing if they earned at least 5 rewards during the last three sessions of the FR3 schedule and the reward delivery was equal distributed over the session. Subsequently, responding for nicotine was extinguished. In this extinction phase (11 daily sessions of 60 min), the animals were placed in the operant chambers, but the nicotine and nicotine related stimuli (discrete and discriminative cues) were not available. Finally, the animals were tested for cue-induced reinstatement. During this test (duration of 30 minutes), the experimental conditions were similar to the conditions during training on an FR3-schedule, with the exception that no nicotine was available and the animals were not connected to the swivel.

Tests for cue-induced reinstatement were conducted twice; between these two test days, there was a week of further extinction training (wash-out period). During cue-induced reinstatement, effects of 5-HT₆ antagonists were studied on output measurements.

Drugs: Nicotine for self-administration: (−) nicotine hydrogen tartrate salt (Sigma, St Louis, Mo.) was dissolved in sterile saline. The pH of the solution was adjusted to +/−7.4 with diluted NaOH. Nicotine dose was expressed as free base weight. Compound 33 was suspended in 5% of end volume Tween 80. Ethanol was added and evaporated under N₂-gas for 30 minutes. Subsequently, 5% of the end volume PEG 400, 25% of the end volume Methylcellulose 1% and 55% of the end volume H₂O HPLC was added which resulted in a homogenous suspension with a pH of 3.76. After the pH was adjusted to 5.5, 10% of end volume H₂O HPLC was added. In the range: 0, 3, 10, and 30 mg/kg, compound 33 was administered intraperitoneally, 15 minutes before testing. The injection volume was 2 ml/kg.

Statistical analysis: For analysis of the cue-induced reinstatement data, the total number of nose-pokes (responses) in the active and inactive hole were subjected to a one-way ANOVA, followed by a Fisher's LSD Multiple-Comparison Test, with the different doses of 5-HT₆ antagonist as between factor. Because each animal was tested twice for cue-induced reinstatement and cue-reactivity, is the results were usually lower during the second test; test day was used as a covariate.

### Pharmacological Test Results

### Cue-Induced Relapse of Alcohol Seeking in the Rat: Compound 33

In the operant cage, all animals readily acquired alcohol self-administration behavior and consumed their rewards. They made a clear distinction between the active and inactive hole. Mean alcohol intake amounted to 0.50 g/kg/hr. During test day's extinction session, responding in the previously active hole extinguished in all of the animals.

| | |
|---|---|
| | compound 33 |
| 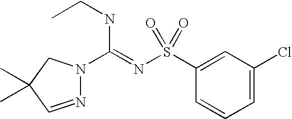 | |

Nose-pokes after an
alcohol induced cue

| treatment groups | former alcoholics | total abstainers |
|---|---|---|
| 0 mg/kg compound 33 | 22.1 ± 4.7 | 1.2 ± 0.6 |
| 3 mg/kg compound 33 | 27.0 ± 6.1 | 1.1 ± 0.2 |
| 10 mg/kg compound 33 | 23.5 ± 4.2 | 2.2 ± 1.5 |
| 30 mg/kg compound 33 | 1.8* ± 0.9 | 0.8 ± 0.1 |

A one-way ANOVA followed by a Fisher's LSD Multiple-Comparison Test revealed that during testing for cue induced alcohol seeking, the amount of active nose-pokes of animals treated with 30 mg/kg of compound 33, was significantly lower than animals treated with placebo. (*p<0.005). Thus, the animals treated with 30 mg/kg of compound 33 were prevented from relapse into addiction. There was no significant difference in inactive nose-pokes between placebo and the tested dosages of compound 33.

### Cue-Induced Relapse of Alcohol Seeking in the Rat: SB-271046

In the operant cage, all animals readily acquired alcohol self-administration behavior and consumed their rewards. They made a clear distinction between the active and inactive hole. Mean alcohol intake amounted to 0.50 g/kg/hr. During test day's extinction session, responding in the previously active hole extinguished in all of the animals.

| | |
|---|---|
| | SB-271046 |
| 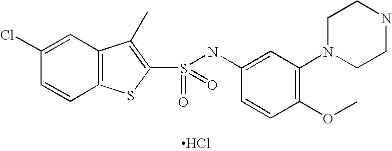 | |

Nose-pokes after an
alcohol induced cue

| treatment groups | former alcoholics | total abstainers |
|---|---|---|
| 0 mg/kg SB-271046 | 17.7 ± 3.3 | 1.5 ± 0.6 |
| 3 mg/kg SB-271046 | 11.9 ± 5.4 | 1.1 ± 0.5 |
| 10 mg/kg SB-271046 | 6.0* ± 2.7 | 0.9 ± 0.5 |
| 20 mg/kg SB-271046 | 1.2** ± 0.7 | 2.7 ± 1.9 |

A one-way ANOVA followed by a Fisher's LSD Multiple-Comparison Test revealed that during testing for cue induced alcohol seeking, the amount of active nose-pokes of animals treated with 10 mg/kg SB-271046, was significantly lower than animals treated with placebo (*p<0.05). The amount of active nose-pokes for animals with a dose of 20 mg/kg of SB-271046 was significantly lower than animals treated with placebo at the level of **p<0.005. Thus, the animals treated with 20 mg/kg of SB-271046 were prevented from relapse into addiction. There was no significant difference in inactive nose-pokes between placebo and the tested dosages of SB-271046.

### Cue-Induced Relapse of Nicotine Seeking in the Rat.

In the operant cage, 27 out of 32 rats met the criterion for nicotine self-administration, and were selected for further testing. The selected animals readily acquired nicotine self-administration behavior, and showed a clear preference for the active nose-poke hole. During extinction sessions, responding in the previously active hole were extinguished in all of the animals.

| | Nose-pokes after a | |
|---|---|---|
| | nicotine induced cue | |
| treatment groups | former nicotine addicts | total abstainers |
| 0 mg/kg compound 33 | 24.50 ± 4.25 | 3.14 ± 0.64 |
| 3 mg/kg compound 33 | 24.83 ± 3.20 | 4.58 ± 1.17 |
| 10 mg/kg compound 33 | 28.50 ± 7.40 | 3.00 ± 1.04 |
| 30 mg/kg compound 33 | 7.92* ± 4.44 | 1.75 ± 0.74 |

A one-way ANOVA followed by a Fisher's LSD Multiple-Comparison Test revealed that during testing for cue induced nicotine seeking, the amount of active nose-pokes of animals treated with 30 mg/kg of compound 33, was significantly lower than animals treated with placebo. (*p<0.05). Thus, the animals treated with 30 mg/kg of compound 33 were prevented from relapse into addiction. There was no significant difference in inactive nose-pokes between placebo and the tested dosages of compound 33.

### Pharmaceutical Preparations

For clinical use, 5-HT₆ antagonists are formulated into pharmaceutical compositions that contain the compounds, more particularly specific compounds, disclosed herein. Types of pharmaceutical compositions that may be used include, but are not limited to, tablets, chewable tablets, capsules (including microcapsules), solutions, parenteral solutions, ointments (creams and gels), suppositories, suspensions, and other types disclosed herein, or are apparent to a person skilled in the art from the specification and general knowledge in the art. The active ingredient for instance, may also be in the form of an inclusion complex in cyclodextrins, their ethers or their esters. The compositions are used for oral, intravenous, subcutaneous, tracheal, bronchial, intranasal, pulmonary, transdermal, buccal, rectal, parenteral or other ways to administer.

The pharmaceutical formulation of the present invention may contain at least one 5-HT₆ antagonist in admixture with a pharmaceutically acceptable adjuvant, diluent and/or carrier. The total amount of active ingredients in the pharmaceutical formulation is in the range of from about 0.1% (w/w) to about 95% (w/w) of the formulation, for example, from 0.5% to 50% (w/w) or from 1% to 25% (w/w) of the formulation.

5-HT₆ antagonists can be brought into forms suitable for administration by means of usual processes using auxiliary substances such as liquid or solid, powdered ingredients, such as the pharmaceutically customary liquid or solid fillers and extenders, solvents, emulsifiers, lubricants, flavorings, colorings and/or buffer substances. Frequently used auxiliary substances include magnesium carbonate, titanium dioxide, lactose, saccharose, sorbitol, mannitol and other sugars or sugar alcohols, talc, lactoprotein, gelatin, starch, amylopectin, cellulose and its derivatives, animal and vegetable oils such as fish liver oil, sunflower, groundnut or sesame oil, polyethylene glycol and solvents such as, for example, sterile water and mono- or polyhydric alcohols such as glycerol, as well as with disintegrating agents and lubricating agents such as magnesium stearate, calcium stearate, sodium stearyl fumarate and polyethylene glycol waxes. The mixture may then be processed into granules or pressed into tablets. A tablet is prepared using the ingredients below:

| Ingredient | Quantity (mg/tablet) |
|---|---|
| COMPOUND No. 33 | 10 |
| Cellulose, microcrystalline | 200 |
| Silicon dioxide, fumed | 10 |
| Stearic acid | 10 |
| Total | 230 |

The components are blended and compressed to form tablets each weighing 230 mg.

The active ingredients may be separately premixed with the other non-active ingredients, before being mixed to form a formulation. The active ingredients may also be mixed with each other, before being mixed with the non-active ingredients to form a formulation.

Soft gelatin capsules may be prepared with capsules containing a mixture of the active ingredients of the invention, vegetable oil, fat, or other suitable vehicle for soft gelatin capsules. Hard gelatin capsules may contain granules of the active ingredients. Hard gelatin capsules may also contain the active ingredients together with solid powdered ingredients such as lactose, saccharose, sorbitol, mannitol, potato starch, corn starch, amylopectin, cellulose derivatives or gelatin.

Dosage units for rectal administration may be prepared (i) in the form of suppositories that contain the active substance mixed with a neutral fat base; (ii) in the form of a gelatin rectal capsule that contains the active substance in a mixture with a vegetable oil, paraffin oil or other suitable vehicle for gelatin rectal capsules; (iii) in the form of a ready-made micro enema; or (iv) in the form of a dry micro enema formulation to be reconstituted in a suitable solvent just prior to administration.

Liquid preparations may be prepared in the form of syrups, elixirs, concentrated drops or suspensions, e.g., solutions or suspensions containing the active ingredients and the remainder consisting, for example, of sugar or sugar alcohols and a mixture of ethanol, water, glycerol, propylene glycol and polyethylene glycol. If desired, such liquid preparations may contain coloring agents, flavoring agents, preservatives, saccharine and carboxymethyl cellulose or other thickening agents. Liquid preparations may also be prepared in the form of a dry powder, reconstituted with a suitable solvent prior to use. Solutions for parenteral administration may be prepared as a solution of a formulation of the invention in a pharmaceutically acceptable solvent. These solutions may also contain stabilizing ingredients, preservatives and/or buffering ingredients. Solutions for parenteral administration may also be prepared as a dry preparation, reconstituted with a suitable solvent before use.

Also provided according to the present invention are formulations and “kits of parts” comprising one or more containers filled with one or more of the ingredients of a pharmaceutical composition of the invention, for use in medical therapy. Associated with such container(s) can be various written materials such as instructions for use, or a notice in the form prescribed by a governmental agency regulating the manufacture, use or sale of pharmaceuticals products, which notice reflects approval by the agency of manufacture, use, or sale for human or veterinary administration.

The use of formulations of the present invention in the manufacture of medicaments for preventing relapse into addiction, and methods of medical treatment or comprising the administration of a therapeutically effective total amount of at least one 5-HT₆ antagonist, either as such or, in the case of prodrugs, after administration, to a patient.

By way of example and not of limitation, several pharmaceutical compositions are provided, comprising examples of active compounds for systemic use or topical application. Other compounds of the invention or combinations thereof, may be used in place of (or in addition to) said compounds. The concentration of the active ingredient may be varied over a wide range as discussed herein. The amounts and types of ingredients that may be included are well known in the art.

### BIBLIOGRAPHY

To the extent in which the following references are useful to one skilled in the art, or to more fully describe this invention, they are incorporated herein by reference. Neither these, nor any other documents or quotes cited herein, nor citations to any references, are admitted to be prior art documents or citations.
Berge, S. M.: “Pharmaceutical salts”, J. Pharmaceutical Science, 66, 1-19 (1977).
Bickel, M. H.,: “The pharmacology and Biochemistry of N-oxides”, Pharmacological Reviews, 21 (4), 325-355, 1969.
Bundgaard, H. (editor), “Design of Prodrugs”, Elsevier, 1985.
Byrn et al., Pharmaceutical Research, 12(7), 945-954, 1995.
Childress, A. R., Ehrman, R., Rohsenow, D. J., Robbins, S. J. and O'Brien, C. P., “Classically conditioned factors in drug dependence”, in: Lowinson, J. W., Luiz, P., Millman, R. B. and Langard, G. (eds.) Substance Abuse: A Comprehensive Textbook pp. 56-69 (Williams and Wilkins, Baltimore, Md., 1992).
De Vries, T. J., Schoffelmeer, A. N. M., Binnekade, R. & Vanderschuren, L. J. M. J. “Dopami-nergic mechanisms mediating the incentive to seek cocaine and heroin following long-term withdrawal of IV drug self-administration”. Psychopharmacology 143, 254-260 (1999).
De Vries, T. J., Shaham, Y., Homberg, J. R., Crombag, H., Schuurman, K., Dieben, J., Vanderschuren, L. J. M. J. and A. N. M. Schoffelmeer, “A cannabinoid mechanism in relapse to cocaine seeking”, Nature Medicine (2001); 7(10): 1151-1154
De Vries, T. J., Homberg, J. R., Binnekade, R., Raaso, H. and A. N. M. Schoffelmeer, “Cannabinoid modulation of the reinforcing and motivational properties of heroin and heroin-associated cues in rats”,Psychopharmacology (Berl) (2003); 168(1-2): 164-9
De Vries, T. J., De Vries, W., Janssen, M. C. W., Schoffelmeer, A. N. M., “Suppression of conditioned nicotine and sucrose seeking by the cannabinoid-1 receptor antagonist SR141716A”, Behavioural Brain Research (2005); 161: 164-168
De Wit, H., “Priming effects with drugs and other reinforcers”, Exp Clin Psychopharmacol (1996); 4: 5-10
Dwyer & Meilor,: “Chelating agents and Metal Chelates”, Academic Press, chapter 7, 1964.
Ettmayer, P. et al., “Lessons learned from marketed and investigational prodrugs”, J. Med. Chem., 47, 2393-2404, 2004.
Järvinen, T. et al., “Design and Pharmaceutical applications of prodrugs”, pages 733-796 in: S. C. Gad (editor): “Drug Discovery Handbook”, John Wiley & Sons Inc., New Jersey, U.S.A., 2005.
King, F. D., (editor), page 215 in: “Medicinal Chemistry: Principles and Practice”, 1994, ISBN 0-85186-494-5.
Lê, A. D., Quan, B., Juzytch, W., Fletcher, P. J., Joharchi, N. and Shaham, Y., “Reinstatement of alcohol-seeking by priming injections of alcohol and exposure to stress in rats”, Psychopharmacology (1998); 135:169-174
Linseman, M. A., “Alcohol consumption in free-feeding rats: procedural, genetic and pharmacokinetic factors”, Psychopharmacology (1987); 92: 254-261
Liu, X. and Weiss, F., “Additive Effect of Stress and Drug Cues on Reinstatement of Ethanol Seeking: Exacerbation by History of Dependence and Role of Concurrent Activation of Corticotropin-Releasing Factor and Opioid Mechanisms”, J. of Neuroscience (2002); 22(18): 7856-7861
Martin, E. W. (Editor), “Remington: The Science and Practice of Pharmacy”, Mack Publishing Company, 19^{th} Edition, Easton, Pa., Vol 2., Chapter 83, 1447-1462, 1995.
Shaham, Y., et al., “The reinstatement model of drug relapse: history, methodology and major findsings”, Psychopharmacology (Berl.), 168,164-169, 2003.
Sinha, R., Catapano, D. and O'Mally, S., “Stress-induced craving and stress responses in cocaine dependent individuals”, Psychopharmacology (1999); 142: 343-351
Stella, J., “Prodrugs as therapeutics”, Expert Opin. Ther. Patents, 14(3), 277-280, 2004.
Tatham, T. A., Zurn, K. R., “The MED-PC experimental apparatus programming system”, Behav Res Meth Instr Comp (1989); 21:294-302

What is claimed is:

1. A method for preventing relapse into addiction comprising administering a pharmaceutical composition comprising a 5 HT₆ antagonist.

2. The method as claimed in claim 1, wherein said 5-HT₆ antagonist is chosen from BGC-20-761, BVT-74316, CNS-10000, CNS-11010, CNS-25100, diF-BAMPI, E-6837, FMPD, GSK-215083, JCF-177, KR-055014, KR-055015, LY-483518, MS-245, PHA-565272A, PRX-07034, Ro 04-6790, Ro-4368554, Ro 63-0563, Ro 65-7199, Ro 65-7674, Ro 66-0074, SAM-315, SB-214111, SB-258510, SB-258585, SB-271046, SB-331711, SB-357134, SB-399885, SB-699929, SB-737050A, SB-742457, ST-1938, and WAY-181187, or a tautomer, stereoisomer, N-oxide or isotopically-labelled analogue thereof, or a pharmacologically acceptable salt, hydrate or solvate of any of the foregoing.

3. The method as claimed in claim 1, wherein said 5-HT₆ antagonist is a compound of formula (1): 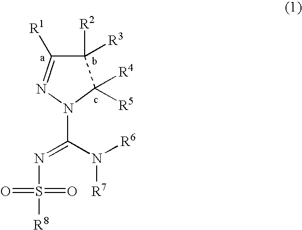
or a tautomer, stereoisomer, N-oxide or isotopically-labelled analogue thereof, or a pharmacologically acceptable salt, hydrate or solvate of any of the foregoing, wherein:
R₁ is chosen from a hydrogen atom, an unsubstituted alkyl(C₁₋₄) group, and an alkyl(C₁₋₄) group substituted with one or more halogen atoms;
R₂ and R₃ are independently chosen from a hydrogen atom, an unsubstituted alkyl(C₁₋₄) group, and an alkyl(C₁₋₄) group substituted with one or more halogen atoms, or,
R₁ and R₂, together with the C-atoms marked ‘a’ and ‘b,’ form a C₅₋₈-cycloalkyl ring, or
R₂ and R₃, together with the carbon atom marked ‘b,’ form a C₃₋₈-cycloalkyl ring;
the dotted line between the carbon atoms marked ‘b’ and ‘c’ represents either a single or a double bond;
R₄ and R₅ are independently chosen from a hydrogen atom, an unsubstituted alkyl(C₁₋₄) group, and an alkyl(C₁₋₄) group substituted with one or more halogen atoms, or,
R₃ and R₄, together with the C-atoms marked ‘b’ and ‘c,’ form a C₃₋₈-cycloalkyl ring, or
R₄ and R₅, together with the carbon atom marked ‘c,’ form a C₃₋₈-cycloalkyl ring;
R₆ and R₇ are independently chosen from a hydrogen atom, an alkyl(C₁₋₄) group, an alkyl (C₁₋₄) group substituted with one or more halogen atoms, a (C₁₋₃)alkoxy group, a dialkyl(C₁₋₃)amino-alkyl(C₁₋₃) group, an optionally substituted aryl group, an optionally substituted C₅₋₈-cyclo-alkyl group, and an optionally substituted C₅₋₈-heterocycloalkyl group, or
R₆ and R₇, together with the nitrogen atom to which they are attached, form an optionally substituted C₅₋₈-heterocycloalkyl group; and
R₈ is chosen from an optionally substituted aryl group, and an —CR₉═CR₁₀-aryl group, wherein R₉ and R₁₀ are independently chosen from a hydrogen atom, an alkyl-(C₁₋₃) group, and a —C≡C-aryl group.

4. The method as claimed in claim 3, wherein
R₁ is a hydrogen atom, or R₁ and R₂, together with the carbon atoms marked ‘a’ and ‘b,’ form a cyclohexyl ring;
R₂ and R₃ are independently chosen from a hydrogen atom, and an alkyl(C₁₋₃) group, or R₂ and R₃, together with the carbon atom marked ‘b,’ form a cyclopentyl or cyclohexyl ring;
the dotted line between the carbon atoms marked ‘b’ and ‘c’ represents either a single or a double bond;
R₄ and R₅ are independently chosen from a hydrogen atom and an alkyl(C₁₋₃) group, or
R₃ and R₄, together with the carbon atoms marked ‘b’ and ‘c,’ form a C₃₋₈-cycloalkyl ring;
R₆ and R₇ are independently chosen from a hydrogen atom, an alkyl(C₁₋₃) group, an alkyl(C₁₋₄) group substituted with one or more halogen atoms, a methoxy group, a cyclohexyl group, a benzyl group, and a 4-piperidinyl group; and
R₈ is chosen from an optionally substituted aryl group, and a —CR₉═CR₁₀-aryl group, wherein R₉ and R₁₀ are independently chosen from a hydrogen atom, an alkyl(C₁₋₃) group, and a —C≡C-aryl group.

5. The method as claimed in claim 4, wherein each of R₁, R₄, R₅ and R₆ is a hydrogen atom, R₂ and R₃ are independently an alkyl(C₁₋₃) group, or R₂ and R₃, together with the carbon atom marked ‘b,’ form a cyclopentyl or cyclohexyl ring, the dotted line between the carbon atoms marked ‘b’ and ‘c’ represents a single bond, R₇ is an alkyl(C₁₋₃) group, and R₈ is a mono- or bicyclic aryl group, substituted with one or more halogen atoms.

6. The method as claimed in claim 3, wherein said 5-HT₆ antagonist is the compound:

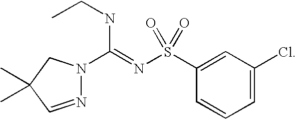

7. The method as claimed in claim 1, wherein said addiction is to substances of abuse, to medicines, or to addictive behaviors.

8. The method as claimed in claim 7, wherein said substances of abuse are chosen from opiates, hallucinogens, inhalants, phencyclidine, amphetamines, cocaine, cannabis, nicotine, and alcohol.

9. The method as claimed in claim 7, wherein said substance of abuse is alcohol.

10. The method as claimed in claim 7, wherein said substance of abuse is nicotine.

11. The method as claimed in claim 7, wherein said substance of abuse is cannabis.

12. The method as claimed in claim 7, wherein said substance of abuse is opiates.

13. The method as claimed in claim 7, wherein said substance of abuse is cocaine.

14. The method as claimed in claim 7, wherein said medicines are chosen from sedatives, hypnotics and anxiolytics.

15. The method as claimed in claim 7, wherein said addictive behavior is gambling.
